(19) United States
(12) Patent Application Publication
(10) Pub. No.: US 2023/0033721 A1
(43) Pub. Date: Feb. 2, 2023

(54) Bridle Delivery System Having Retrieval Probe with Flexible Distal Tip

(71) Applicant: Avent, Inc., Alpharetta, GA (US)

(72) Inventors: Sarah B. Kokotoff, Alpharetta, GA (US); Vernon Meadows, Lilburn, GA (US)

(21) Appl. No.: 17/386,713

(22) Filed: Jul. 28, 2021

(51) Int. Cl. A61M 25/02; A61M 25/00; A61M 25/01

(52) U.S. Cl. CPC A61M 25/02; A61M 25/0043; A61M 25/0158; A61M 2205/0216; A61M 2025/0059; A61M 2025/0226

(57) ABSTRACT

A retrieval probe for insertion of a bridle into a nasal passageway of a patient is provided. The retrieval probe includes a proximal end and a distal end, a catheter between the proximal end and the distal end, an inner core within a lumen of the catheter, and a magnetic tip disposed at the proximal end. The catheter includes a hollow section disposed between the magnetic portion and a distal end of the stiff inner core. The hollow section may be flexible and can be configured to bend at an angle of about 180 degrees. The system for securing a nasal tube can include a bridle having a magnetic connection portion; and a retrieval probe as described above. The magnetic connection portion of the bridle and the magnetic tip of the retrieval probe can be configured to magnetically couple during a procedure for inserting the bridle in a nasal passageway of a patient.

### FIELD OF THE INVENTION

The subject matter of the present invention relates generally to a system for securing a nasal tube including a retrieval probe with a flexible distal tip for insertion of a bridle into a nasal passageway of a patient.

### BACKGROUND

The use of nasal tubes is commonly required in a medical setting, and many methods of securing nasal tubes that have been placed are known in the art. Generally, a nasal tube which has been inserted into a nostril may extend into a patient's stomach, intestinal tract, or lungs. Typically, once the nasal tube is in place, it is important to secure the tube. It should be appreciated that failing to properly secure a nasal tube can result in a dangerous situation for a patient, as well as increasing the cost of care, for example, by requiring repositioning of the nasal tube and re-securing the nasal tube.

There are various existing systems and methods for securing a nasal tube. For example, a nasal tube may be secured using a bridle being placed around the vomer bone. A clinician may place the bridle using a long flexible member, such as a tube, including the bridle and a magnet at the distal end, which is held together by the clinician's grasp. The long flexible member is inserted into one nostril, into the naval cavity towards the rear of the vomer bone. A probe with a magnet at the distal end is inserted into the other nostril to allow the magnets to contact each other around the vomer bone. Once the magnets have made contact, the clinician lets go of the bridle to allow the long flexible member to enter the nostril, and the probe is pulled outward, which pulls the long flexible member including the bridle around the vomer bone. With the bridle looped around the vomer bone and extending from both nostrils, the bridle may be secured with a clamp, which may have a channel to accept the nasal tube to secure the nasal tube. For example, the channel can have a smaller inside diameter than the outside diameter of the nasal tube, which provides for a tight fit of the nasal tube in the channel and allows the nasal tube to not fall out of the clamp prior to closing the clamp. The clinician may bring the clamp as close to the nostril as possible and press the tube into the tight channel in the clamp, place the bridle into the clamp, and close the clamp to secure the nasal tube to the bridle.

Existing nasal bridling devices for nasal tube securement typically rely on two catheters inserted into the nares past the vomer bone with magnets at the tip of each catheter to facilitate connection between the catheters behind the vomer bone and pass through of a tether through both nares. These catheters are stiff in nature and connection is made behind the vomer bone at an angle relying on magnetic attraction rather than catheter flexion to make the catheters connect. The catheters must be stiff enough to prevent the catheters from collapsing while entering the nasal passageway and yet flexible enough to curve behind the vomer bone to aid in connection of the catheters. Often, the catheters include a coil wire to increase the stiffness of the catheter, which can result in kinking of the coil wire if the catheter is bent. As a result, the tradeoffs between stiffness and flexibility of the catheters result in increased opportunity for misconnection between the catheters. Moreover, the magnets on these current devices are often limited in magnet exposure. For instance, in some devices the magnet is completely encapsulated in the catheter. Thus, there may be a very small margin for error while connecting the catheters, which more opportunities for misconnection as the connecting surfaces must be near perfect alignment to connect. The increased opportunity for misconnection between the catheters can result in repeated or lengthened procedure time, increasing monetary cost, time, and pain for patients.

Consequently, there is a need for an improved bridle delivery system to allow for controlled insertion into the nasal passageway while providing flexibility to curve around the back of the vomer bone. In particular, an improved bridle delivery system that reduces the probability for misconnections would also be useful.

### SUMMARY

Objects and advantages of the invention will be set forth in part in the following description, or may be obvious from the description, or may be learned through practice of the invention.

The present invention is directed to a retrieval probe for insertion of a bridle into a nasal passageway of a patient. The retrieval probe includes a proximal end and a distal end; and a catheter between the proximal end and the distal end. The retrieval probe further includes a stiff inner core within a lumen of the catheter, and a magnetic tip disposed at the proximal end. The catheter includes a hollow section disposed between the magnetic tip and a distal end of the stiff inner core.

In one particular embodiment, the catheter can include a first material and the inner core can include a second material. Moreover, the first material and the second material can be dissimilar. Furthermore, the first material can have a first durometer and the second material can have a second durometer, wherein a ratio of the first durometer to the second durometer is in a range from about 1:2 to about 1:4. Moreover, the first material can be a thermoplastic elastomer and the second material can be nylon.

In another embodiment, the inner core can be formed from a solid construction.

In an additional embodiment, the hollow section can be configured to bend at an angle of about 180 degrees without kinking.

In a further embodiment, the hollow section can include a length in a range from about 1.5 cm to about 2.5 cm.

In yet another embodiment, the magnetic tip can include a magnetic holding tip and a connection member. Moreover, a proximal end of the connection member can be disposed within the magnetic holding tip and a distal end of the connection member can protrude from the magnetic holding tip. Further, the connection member can include an exposed section extending from a distal end of the magnetic holding tip to a distal end of the connection member. Moreover, the exposed section can have a length in a range from about 0.75 mm to about 2 mm. Further, a ratio of a length of the exposed section to a length of the connection member can be in a range from about 1:5 to about 1:2. Moreover, a proximal end of the magnetic holding tip can be disposed within the distal end of the catheter. Further, the connection member can include a permanent magnet and/or a magnetically connective material.

The present invention is further directed to a system for securing a nasal tube. The system includes a bridle, a magnetic connection portion attached to the bridle, and a retrieval probe. The retrieval probe includes a proximal end and a distal end; a catheter between the proximal end and the distal end; an inner core within a lumen of the catheter; and a magnetic tip disposed at the proximal end. The catheter comprises a hollow section disposed between the magnetic tip and a distal end of the inner core. Further, the magnetic connection portion of the bridle and the magnetic tip of the retrieval probe are configured to magnetically couple during a procedure for inserting the bridle in a nasal passageway of a patient.

In one aspect of the system, either the magnetic connection portion of the bridle or the magnetic tip of the retrieval probe can include a permanent magnet. Moreover, the other of the magnetic connection portion of the bridle or the magnetic tip of the retrieval probe can include a magnetically connective material that is not a permanent magnet.

In another aspect of the system, the magnetic connection portion of the bridle and the magnetic tip of the retrieval probe each can include a permanent magnet.

In another aspect of the system, the hollow section of the retrieval probe can be configured to bend up to about 180 degrees to enable coupling of the magnetic connection portion of the bridle and the magnetic tip of the retrieval probe during the procedure for inserting the bridle.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a side view of a retrieval probe of a system for securing a nasal tube according to one particular embodiment of the present invention;

FIG. 2 illustrates a cross-sectional view of the retrieval probe of FIG. 1 taken along the line 2-2;

FIG. 3 illustrates a detailed cross-sectional view of a distal end of the retrieval probe of FIG. 1;

FIG. 4 illustrates a detailed cross-sectional view of a proximal catheter transition section of the retrieval probe of claim 1; and

FIG. 5 illustrates a perspective view of a nasal bridle of the system for securing a nasal tube according to the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

Generally speaking, the present invention is directed to a retrieval probe for insertion of a bridle into a nasal passageway of a patient and a system for securing a nasal tube. The retrieval probe includes a proximal end and a distal end, and a catheter between the proximal end and the distal end. The retrieval probe also includes an inner core within a lumen of the catheter and a magnetic tip disposed at the proximal end, such that the catheter comprises a hollow section disposed between the magnetic portion and a distal end of the inner core. The magnetic tip includes a connection member formed from a magnetically connective material that is exposed on a distal surface and a lateral surface at the distal end of the retrieval probe. The present inventors have found that providing an inner core within the lumen of the catheter and a hollow section disposed between the magnetic portion and a distal end of the inner core enables the retrieval probe to have significantly improved flexibility at the distal end of the retrieval probe while maintaining increased rigidity control of the length of the retrieval probe proximal to the hollow section while inserting the retrieval probe into a patient's nasal passageway. Moreover, the present inventors have found that the exposure of the magnetically connective connection member at the distal end of the retrieval probe increases the exposure of a magnetic surface to facilitate a stronger connection between the retrieval probe and a coupling member, such as a securement device, without sacrificing patient safety.

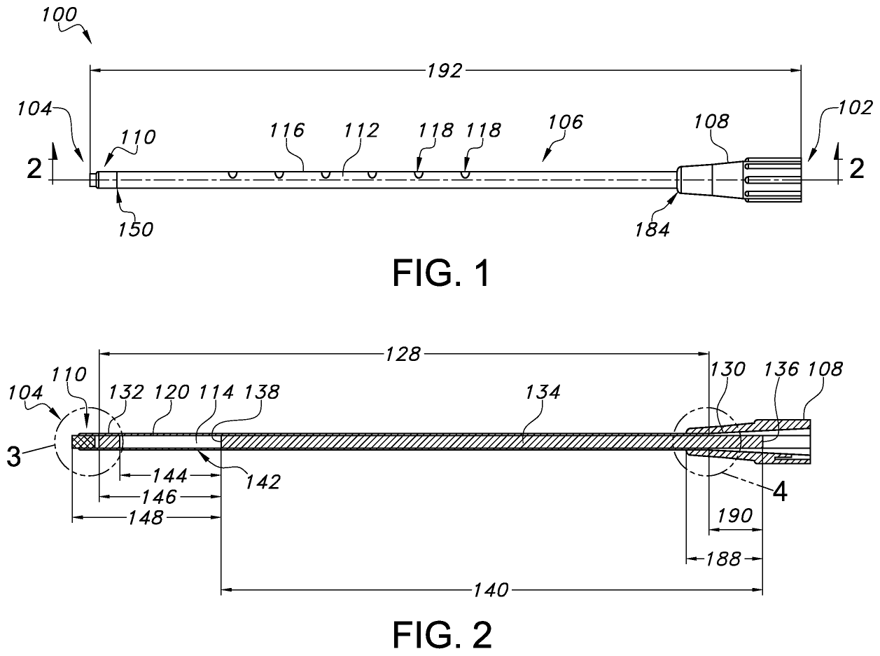

The specific features of the retrieval probe and system for securing a nasal tube of the present invention may be better understood with reference to FIGS. 1-5.

Referring now to FIG. 1, one embodiment of a retrieval probe 100 for a system for securing a nasal tube is shown. The retrieval probe 100 extends from a proximal end 102 to a distal end 104. The retrieval probe 100 includes an elongated member 106 that extends between the proximal end 102 and the distal end 104.

The retrieval probe 100 includes a handle 108 at the proximal end 102, and a magnetic tip 110 at the distal end 104. The elongated member 106 of the retrieval probe 100 may be approximately 5 inches (about 12.7 cm) long, with the handle 108 being approximately 1 inch (about 2.54 cm) long.

The elongated member 106 can include a flexible catheter 107 formed from a catheter wall 112 surrounding a lumen 114. The catheter 107 has a length 128 extending from a proximal end 130 of the catheter 107 to a distal end 132 of the catheter 107. The length 128 can be in a range from about 4 inches (about 10 cm) to about 6 inches (about 15 cm), such as from about 4.5 inches (about 11.4 cm) to about 5.5 inches (about 14 cm), such as about 5 inches (about 12.7 cm).

The catheter wall 112 has an outer surface 116 and an inner surface 120. Optionally, as shown in FIG. 1, the outer surface 116 can include one or more markings 118 to allow a clinician to easily see how far into the nasal cavity the retrieval probe 100 has been inserted. For instance, the markings 118 may include indices (e.g., 1 cm through 9 cm) analogous to a ruler.

The catheter 107 can have an outer diameter 124 defined by the outer surface 116 and an inner diameter 126, i.e., a diameter of the lumen 114, defined by the inner surface 120 of the catheter wall 112. The outer diameter 124 can be in a range from about 0.05 inches (about 1.3 mm) to about 0.17 inches (about 4.3 mm), such as from about 0.08 inches (about 2 mm) to about 0.15 inches (about 3.8 mm), such as from about 0.10 inches (about 2.6 mm) to about 0.13 inches (about 3.3 mm). To define the outer diameter 124 in other terms, the catheter 107 can be in a size from 4 French to 13 French. The inner diameter 126 of the catheter 107 can be in a range from about 0.04 inches (about 1 mm) to about 0.1 inches (about 2.5 mm), such as from about 0.06 inches (about 1.6 mm) to about 0.09 inches (about 2.3 mm), for instance about 0.08 inches (about 2 mm). The catheter wall 112 has thickness 122 in the radial direction from the outer surface 116 to the inner surface 120. The thickness 122 can be in a range from about 0.01 inches (about 0.25 mm) to about 0.03 inches (about 0.75 mm).

The catheter 107 can be formed from a soft, pliable material. For instance, the wall 112 of the catheter 107 can be formed from a variety of materials, giving due consideration to the goals of flexibility, lightweight, strength, smoothness, and biocompatibility or non-reactivity to anatomical systems, i.e., safety. Suitable materials for the catheter 107 include thermoplastic elastomers such as polyolefins, including polyethylene and polypropylene, polyamides, polyimides, teflon (polytetrafluoroethylene), polyesters, polyurethanes, any copolymers thereof, and other materials known in the art. In some particular embodiments, the catheter 107 can be formed from polyether block amide (PEBA), which may be sold under the trade name PEBAX®. The material of the catheter 107 can have a durometer in a range from about 15 to about 35, such as from about 20 to about 30, such as a durometer of about 25.

An inner core 134 can be provided to the catheter 107. For instance, as shown in FIGS. 2 and 4, the inner core 134 can be disposed within the lumen 114 of the catheter 107, such as being inserted within the lumen 114. The inner core 134 can extend from a proximal end 136 to a distal end 138, having a length 140 extending from the proximal end 136 to the distal end 138 of the inner core 134. Generally, the length 140 of the inner core 134 can be shorter than a length 128 of the catheter 107 and/or the inner core 134 may partially extend from an end of the catheter 107, as described in further detail below. The length 140 can be in a range from about 4 inches (about 10.2 cm) to about 5 inches (about 12.7 cm), such as from about 4.2 inches (about 10.7 cm) to about 4.7 inches (about 11.9 cm), such as from about 4.3 inches (about 10.9 cm) to about 4.5 inches (about 11.4 inches). When disposed within the catheter 107, the inner core 134 provides rigidity to the catheter 107 to improve control of the catheter 107, e.g., during an insertion procedure. The inner core 134 can be inserted within the lumen 114 of the catheter 107. The diameter 192 can be in a range from about 0.03 inches (about 0.75 mm) to about 0.09 inches (about 2.3 mm), such as from about 0.05 inches (about 1.3 mm) to about 0.08 inches (about 2 mm), for instance about 0.075 inches (about 1.9 mm).

The inner core 134 can be formed from a material having a higher degree of stiffness or rigidity and low flexibility compared to the catheter 107. For instance, the inner core 134 can be formed from nylon or any other suitable material. The material of the inner core 134 can be dissimilar from the material of the catheter 107. In some aspects of the invention, the inner core 134 is formed from a solid piece of material (“solid construction”), e.g., extruded in a generally cylindrical shape. The inner core 134 can have a diameter 192 small enough to be disposed within the lumen 114 of the catheter 107 while still providing sufficient stiffness and support to the flexible catheter 107. The material of the inner core 134 can have a durometer of in a range from about 60 to about 90, such as from about 70 to about 80, such as a durometer of about 75 shore D. In this regard, a ratio of the durometer of the catheter 107 material to the durometer of the inner core 134 can be in a range from about 1:2 to about 1:4, such as about 1:3. The ratio of the durometer of the catheter 107 material to the durometer of the inner core 134, along with the wall thickness 122 as described above, can be selected to optimize the flexibility and control of the elongated member 106. For instance, the durometer of the inner core 134 can be reduced to increase the flexibility of the elongated member 106.

As shown in FIG. 2, when the inner core 134 is disposed within the catheter 107 of the retrieval probe 100, the distal end 138 of the inner core 134 can be disposed proximal to the distal end 132 of the catheter 107. A hollow section 142 of the catheter 107 can be formed toward the distal end 132 of the catheter 107. The hollow section 142 can form a flexible portion of the catheter 107. The hollow section 142 can have a length 146 extending from the distal end 132 of the catheter 107 to the distal end 138 of the inner core 134 within the lumen 114 of the catheter 107. The length 146 can be in a range from about 0.5 inches (about 1.25 cm) to about 2 inches (about 5 cm), such as from about 0.75 inches (about 1.9 cm) to about 1.5 inches (about 3.8 cm), such as from about 0.9 inches (about 2.3 cm) to about 1.1 inches (about 2.8 cm). In a particular embodiment of the present invention, the length 146 of the hollow section 142 can be about 1 inch (about 2.5 cm). The flexible portion formed by the hollow section 142 can be configured to bend or flex up to about 180 degrees of motion without kinking. For the purpose of the present invention, “kinking” means forming a sharp twist or curve in the catheter 107 that causes a change in the geometry of the lumen 114 and/or the outer surface 116 of the catheter 107 at a particular location. Moreover, the hollow section 142 of the catheter 107 has no memory. In other words, after the hollow section 142 is bent or flexed into a deformed state, the hollow section 142 can return to its original state with no memory of the deformed state. Thus, the hollow section 142 can be bent or flexed around the vomer bone during an insertion procedure with up to about 180 degrees of motion without kinking, thereby enhancing the ease with which a connection can be made with a bridle or other device inserted within the other nare.

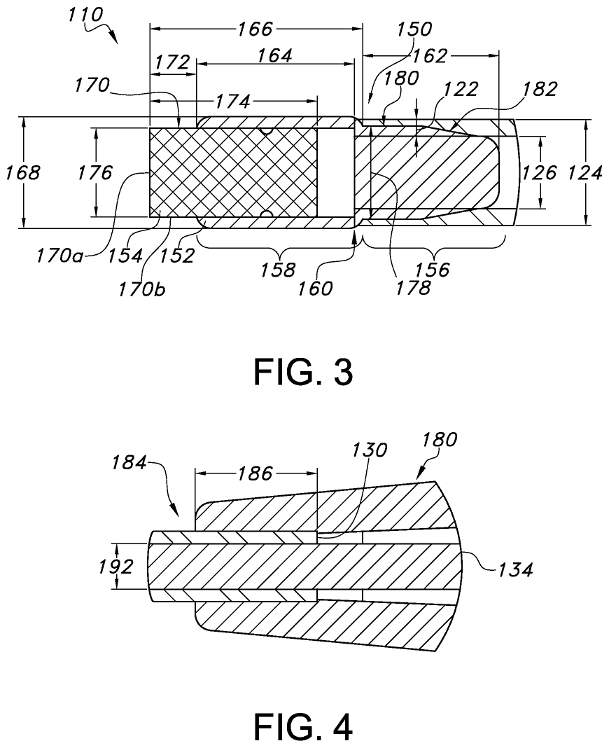

As shown in FIG. 2 and illustrated in detail in FIG. 3, a magnetic tip 110 can be disposed at the distal end 104 of the retrieval probe 100. For instance, the magnetic tip 110 can be disposed adjacent to the distal end 132 of the catheter 107. The magnetic tip 110 can include magnetic holding tip body 152 having a catheter connection section 156 at a proximal end and a protruding section 158 distal to the catheter connection section 156. The catheter connection section 156 can be configured for insertion within the lumen 114 of the catheter 107 at the distal end 132 of the catheter. The protruding section 158 can be configured to extend beyond the distal end 132 of the catheter 107. A shoulder 160 can be formed where the catheter connection section 156 and the protruding section 158 meet. The shoulder 160 can have a radius of curvature extending between the catheter connection section 156 and the protruding section 158 to provide a generally smooth transition and a smooth outer surface of the protruding section 158.

The catheter connection section 156 can have a constant diameter portion 180 having a diameter 178 adjacent to the shoulder 160, and a tapered portion 182 extending between a proximal end of the magnetic holding tip body 152 and the constant diameter portion 180. The catheter connection section 156 can have an interference fit with the inner surface 120 of the catheter wall 112. For instance, the diameter 178 can be in a range from about 0.085 inches (about 2.2 mm) to about 0.115 inches (about 2.9 mm), such as from about 0.095 inches (about 2.4 mm) to about 0.11 inches (about 2.8 mm), such as from about 0.099 inches (about 2.5 mm) to about 0.105 inches (about 2.7 mm). The catheter connection section 156 can have a total length in a range from about 0.15 inches (about 3.8 mm) to about 0.2 inches (about 5.1 mm), such as from about 0.16 inches (about 4 mm) to about 0.18 inches (about 4.6 mm).

In other words, the catheter connection section 156 can extend a distance approximately equal to the length into the lumen 114 of the catheter 107 at the distal end 132 of the catheter. As a result, the hollow section 142 of the catheter 107 can be formed between the inner core 134 and the magnetic holding tip body 152. Thus, the total length 144 of the hollow section 142 can be in a range from about 0.3 inches (about 0.75 cm) to about 1.85 inches (about 4.7 cm), such as from about 0.55 inches (about 1.4 cm) to about 1.35 inches (about .4 cm), such as from about 0.6 inches (about 1.5 cm) to about 1 inch (about 2.5 cm). As described above, the hollow section 142 may be sufficiently flexible, including having a sufficient length 144 as set forth in the preceding sentence, to bend or flex up to about 180 degrees of motion in any direction.

The protruding section 158 can have a diameter 168 approximately equal to or slightly larger than the outer diameter 124 of the catheter 107. For instance, the diameter 168 can be in a range from about 0.115 inches (about 2.9 mm) to about 0.13 inches (about 3.3 mm), such as from about 0.12 inches (about 3 mm) to about 0.13 inches (about 3.3 mm), for instance about 0.125 inches (about 3.175 mm). In this regard, the protruding section 158 can have a minimally obstructive transition from the catheter 107 to the magnetic tip 110 to reduce opportunity for trauma to a patient's tissue during an insertion procedure. Additionally, the protruding section 158 can have a length 164 from the shoulder 160 to a distal end of the magnetic holding tip body 152 in a range from about 0.15 inches (about 4 mm) to about 0.25 inches (about 6.4 mm), such as from about 0.17 inches (about 4.3 mm) to about 0.2 inches (about 5 mm), for instance from about 0.18 inches (about 4.6 mm) to about 0.19 inches (about 4.8 mm).

The magnetic tip 110 can also include a connection member 154, e.g., a magnet or a magnetically connective material. For instance, the connection member 154 can be formed from a permanent magnet, a rare earth magnet, or any suitable material to provide for magnetic coupling with a cooperating magnetic coupling. The connection member 154 can be held within the magnetic holding tip 152. For instance, the connection member 154 can be disposed within the protruding section 158 of the magnetic holding tip body 152. In some aspects of the present invention, the magnetic holding tip body 152 can be overmolded over the connection member 154 to permanently couple the magnetic holding tip body 152 and the connection member 154; however, any suitable method of attaching the connection member 154 can be used. The connection member 154 is configured to extend or protrude from the magnetic holding tip body 152 as shown in FIGS. 2-3. The connection member 154 can have a generally cylindrical shape having a diameter 176. The diameter 176 can be in a range from about 0.09 inches (about 2.3 mm) to about 0.11 inches (about 2.8 mm), such as about 0.1 inches (about 2.5 mm). The connection member 154 can have an overall length 174 in a range from about 0.12 inches (about 3.05 mm) to about 0.25 inches (about 6.35 mm), such as from about 0.15 inches (about 3.81 mm) to about 0.2 inches (about 5.08 mm), for instance, from about 0.18 inches (about 4.57 mm) to about 0.192 inches (about 4.88 mm).

As shown in FIG. 3, the connection member 154 can have an exposed section 170 that extends or protrudes from the magnetic holding tip body 152 at the distal end 104 of the retrieval probe 100. The exposed section 170 can have a distal surface 170a and a lateral surface 170b. The exposed section 170 can have an axial length 172 along the lateral surface 170b in a range from about 0.03 inches (about 0.6 mm) to about 0.1 inches (about 2.5 mm), such as from about 0.04 inches (about 1 mm) to about 0.07 inches (about 1.8 mm), such as from about 0.045 inches (about 1.1 mm) to about 0.055 inches (about 1.4 mm). A ratio of the length 172 of the exposed section 170 to the overall length 174 of the connection member 154 can be in a range from about 1:5 to about 1:2, for instance from about 1:4 to about 1:3. Notably, the length 172 of the exposed section 170 may be generally about two to three times larger than a length of an exposed section of magnet of prior art retrieval devices. The present inventors have found that the increased length of the exposed section 170 of the connection member 154 of the present invention allows for overall increased exposure of the magnetically connective surface of the connection member 154 along the lateral surface 170b, thereby facilitating a stronger magnetic connection between the retrieval probe 100 and a cooperating device, such as a securement device, without compromising patient safety.

Turning now to FIG. 4, a detailed view of the proximal catheter transition section 184 of the retrieval probe 100 is shown. The proximal end 130 of the catheter 107 is disposed within a lumen of the handle 108. A length 186 of the catheter 107 adjacent to the proximal end 130 extends within the handle 108. The length 186 can be in a range from about 0.1 inches (about 2.5 mm) to about 0.4 inches (about 10 mm), such as from about 0.15 inches (about 4 mm) to about 0.25 inches (about 6.4 mm), for instance from about 0.18 inches (about 4.6 mm) to about 0.22 inches (about 5.6 mm). The catheter 107 can be attached to the handle 108 with an adhesive, such as a cyanoacrylate adhesive, ethyl-based adhesive, or any suitable adhesive or other suitable attachment means.

At the proximal catheter transition section 184, the inner core 134 can be disposed within the catheter 107. For instance, a length 188 of the inner core 134 can extend within the handle 108. As shown in FIG. 2, the inner core 134 can extend within the handle 108 beyond the proximal end 130 of the catheter 107 in a direction toward the proximal end 102 of the retrieval probe 100. The length 188 can be in a range from about 0.5 inches (about 12.7 mm) to about 1 inch (about 25.4 mm), such as from about 0.6 inches (about 15 mm) to about 0.75 inches (about 19 mm), such as from about 0.65 inches (about 16.5 mm) to about 0.7 inches (about 17.8 mm). Moreover, the inner core 134 can extend beyond the proximal end 130 of the catheter 107 in the direction of the proximal end 102 of the retrieval probe 100 a length 190. The length 190 can be in a range from about 0.4 inches (about 10 mm) to about 0.8 inches (about 20 mm), such as from about 0.45 inches (about 11 mm) to about 0.65 inches (about 16.5 mm), such as from about 0.5 inches (about 12.7 mm) to about 0.6 inches (about 15 mm). By extending the inner core 134 in the proximal direction beyond the proximal end 130 of the catheter 107, the present inventors have found that the stiffness of the catheter 107 can be maintained adjacent to the handle 108 for improved control of the retrieval probe 100 during an insertion procedure.

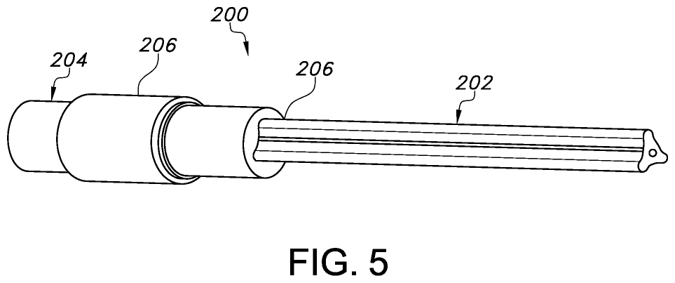

The present invention is further directed to a system for securing a nasal tube that can include the retrieval probe 100 and a securement device, such as the securement device 200 illustrated in FIG. 5. However, it is to be understood that the retrieval probe 100 can be configured for use in a system for securing a nasal tube with any suitable bridle or other securing device having a magnetic coupling means. The securement device 200 can include a bridle 202. The bridle 202 can be an elongated piece of material configured to be threaded through the nares and around the vomer bone to secure a nasal tube. The securement device 200 can further include a magnetic connection portion 204 at a first end 206 of the bridle 202. As shown in FIG. 5, a holding tip 208 can be attached at the first end 206 of the bridle to secure the magnetic connection portion 204 in place. The magnetic connection portion 204 can be formed from a permanent magnet, a rare earth magnet, or any suitable material to provide for magnetic coupling with a cooperating magnetic coupling. For instance, when the retrieval probe 100 includes a connection member 154 formed from a permanent magnet, the magnetic connection portion 204 can be formed from a permanent magnet having opposite polarity, or a material configured to magnetically couple to a permanent magnet that is not a permanent magnet. An example of such material can be stainless steel or any other suitable material having low magnetic reluctance. Alternatively, when the magnetic connection portion 204 includes a permanent magnet, the connection member 154 of the retrieval probe of the present invention can be formed from a material configured to magnetically couple to a permanent magnet that is not a permanent magnet, such as stainless steel.

In use, the securement device 200 can be inserted into a first nostril, e.g., by inserting the securement device within a delivery probe (not shown) and inserting the delivery probe into the first nostril. The magnetic connection portion 204 of the securement device 200 can be inserted towards the rear of the vomer bone of a patient through the first nostril. Then, the retrieval probe 100 can be inserted into a second nostril, with the connection member 154 exposed at the distal end 104 of the retrieval probe 100 and inserted towards the rear of the vomer bone of the patient within the second nostril. The magnetic connection portion 204 of the securement device 200 and the connection member 154 of the retrieval probe 100 can be magnetically coupled behind the patient's vomer bone. For instance, the hollow section 142 of the retrieval probe 100 can bend or flex behind the vomer bone as the magnetic attraction between the connection member 154 and the magnetic connection portion 204 increases to bring the connection member 154 and the magnetic connection portion 204 together. After the magnetic connection portion 204 and the connection member 154 are coupled, the retrieval probe 100 is removed from the second nostril, such that the bridle 202 extends into the first nostril and out from the second nostril. If applicable, the delivery probe is then removed from the first nostril. For example, when the retrieval probe 100 is removed from the second nostril, the bridle 202 is left hanging out of both nostrils. Then, the bridle 202 can be secured using any suitable means. Moreover, a nasal tube can be inserted and/or secured to the bridle using any suitable means to secure the nasal tube within the patient's nose and deter or discourage the patient from pulling on the nasal tube.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A retrieval probe for insertion of a bridle into a nasal passageway of a patient, the retrieval probe comprising:
a proximal end and a distal end;
a catheter between the proximal end and the distal end;
a stiff inner core within a lumen of the catheter;
a magnetic tip disposed at the proximal end;
wherein the catheter comprises a hollow section disposed between the magnetic tip and a distal end of the stiff inner core.

2. The retrieval probe of claim 1, wherein the catheter comprises a first material and the inner core comprises a second material.

3. The retrieval probe of claim 2, wherein the first material and the second material are dissimilar.

4. The retrieval probe of claim 2, wherein the first material has a first durometer and the second material has a second durometer, wherein a ratio of the first durometer to the second durometer is in a range from about 1:2 to about 1:4.

5. The retrieval probe of claim 2, wherein the first material is a thermoplastic elastomer and the second material is nylon.

6. The retrieval probe of claim 1, wherein the inner core is formed from a solid construction.

7. The retrieval probe of claim 1, wherein the hollow section is configured to bend at an angle of about 180 degrees without kinking.

8. The retrieval probe of claim 1, wherein the hollow section comprises a length in a range from about 1.5 cm to about 2.5 cm.

9. The retrieval probe of claim 1, wherein the magnetic tip comprises a magnetic holding tip and a connection member.

10. The retrieval probe of claim 9, wherein a proximal end of the connection member is disposed within the magnetic holding tip and a distal end of the connection member protrudes from the magnetic holding tip.

11. The retrieval probe of claim 10, wherein the connection member comprises an exposed section extending from a distal end of the magnetic holding tip to a distal end of the connection member.

12. The retrieval probe of claim 11, wherein the exposed section comprises a length in a range from about 0.75 mm to about 2 mm.

13. The retrieval probe of claim 11, wherein a ratio of a length of the exposed section to a length of the connection member is in a range from about 1:5 to about 1:2.

14. The retrieval probe of claim 9, wherein a proximal end of the magnetic holding tip is disposed within the distal end of the catheter.

15. The retrieval probe of claim 9, wherein the connection member comprises a permanent magnet and/or a magnetically connective material.

16. A system for securing a nasal tube comprising:
a bridle;
a magnetic connection portion attached to the bridle; and
a retrieval probe, the retrieval probe comprising:
a proximal end and a distal end;
a catheter between the proximal end and the distal end;
an inner core within a lumen of the catheter;
a magnetic tip disposed at the proximal end;
wherein the catheter comprises a hollow section disposed between the magnetic tip and a distal end of the inner core;
wherein the magnetic connection portion of the bridle and the magnetic tip of the retrieval probe are configured to magnetically couple during a procedure for inserting the bridle in a nasal passageway of a patient.

17. The system of claim 16, wherein either the magnetic connection portion of the bridle or the magnetic tip of the retrieval probe comprises a permanent magnet.

18. The system of claim 17, wherein the other of the magnetic connection portion of the bridle or the magnetic tip of the retrieval probe comprises a magnetically connective material that is not a permanent magnet.

19. The system of claim 16, wherein the magnetic connection portion of the bridle and the magnetic tip of the retrieval probe each comprises a permanent magnet.

20. The system of claim 16, wherein the hollow section of the retrieval probe is configured to bend up to about 180 degrees to enable coupling of the magnetic connection portion of the bridle and the magnetic tip of the retrieval probe during the procedure for inserting the bridle.
